(19) United States
(12) Patent Application Publication
(10) Pub. No.: US 2009/0053741 A1
(43) Pub. Date: Feb. 26, 2009

(54) ASSAY FOR MEASURING ASYMMETRIC METHYLARGININE IN A BIOLOGICAL SAMPLE

(76) Inventors: Patrick John Thompson Vallance, London (GB); James Mitchell Leiper, London (GB); David Edward Williams, Auckland (NZ)

Correspondence Address: FULBRIGHT & JAWORSKI, LLP, 1301 MCKINNEY, SUITE 5100, HOUSTON, TX, 77010-3095, US

(21) Appl. No.: 11/910,107

(22) PCT Filed: Mar. 28, 2006
(86) PCT No.: PCT/GB2006/001130

§ 371 Date: Jun. 24, 2008

Related U.S. Application Data
(60) Provisional application No. 60/666,452, filed on Mar. 30, 2005.

(51) Int. Cl. G01N 33/573; C12M 1/00

(52) U.S. Cl. 435/7.72; 435/287.1

(57) ABSTRACT

The invention relates to a method of determining the presence and/or amount of an asymmetric methylarginine in a sample, the method comprising:
(a) contacting the sample with a nitric oxide synthase (NOS) polypeptide in the presence of a detectably labelled species under conditions which permit the asymmetric methylarginine and detectably labelled species to bind to the NOS polypeptide; and
(b) determining the amount or presence of the detectably labelled species bound to the NOS polypeptide

The present invention claims priority to PCT/GB2006/001130, filed Mar. 28, 2006, which claims priority to U.S. Provisional Patent Application Ser. No. 60/666,452, filed Mar. 30, 2005, both of which applications are incorporated by reference herein in their entirety.

### FIELD OF THE INVENTION

The present invention relates to a method of determining the presence and/or amount of an asymmetric methylarginine in a sample and to associated kits.

### BACKGROUND TO THE INVENTION

Asymmetric methylarginines are endogenous inhibitors of nitric oxide synthase (NOS) that compete with binding of the natural substrate L-arginine. They are produced from methylated arginine residues in proteins by protein methyltransferases (PRMT) and are metabolised by the enzyme dimethylarginine dimethylaminohydrolase (DDAH). There are two broad types of PRMTs: type 1 catalyzes the formation of asymmetric dimethylarginine (ADMA) and type 2 catalyzes the formation of symmetric dimethylarginine (SDMA). SDMA does not inhibit NOS. Both types of PRMT can also produce another asymmetric methylarginine, N^{G}-monomethyl-L-arginine (L-NMMA). ADMA and L-NMMA are equipotent at inhibiting NOS.

Plasma concentrations of ADMA have been implicated as a marker of risk for endothelial dysfunction and cardiovascular disease (Vallance and Leiper, Arterioscler. Thromb. Vasc. Biol. 2004; 24: 1-9; and Vallance, The Lancet 2001; 358: 2096-2097). Increased plasma AMDA has been detected in renal failure, type 2 diabetes, heart failure, pre-eclampsia, pulmonary hypertension and various cardiovascular risk factors such as hypercholesterolaemia, hypertension, diabetes, hyperhomocystinaemia and overt atherosclerosis (Vallance and Leiper, Arterioscler. Thromb. Vasc. Biol. 2004; 24: 1-9; and Vallance, The Lancet 2001; 358: 2096-2097). There is therefore a need for a simple, reliable assay of ADMA and other asymmetric methylarginines that can be carried out near a patient.

Previously, High Performance Liquid Chromatography (HPLC) separation and fluorescence detection has been used to measure ADMA. However, this method is complicated and its accuracy and precision are variable. Hence, HPLC is not suited as a simple, reliable assay method for use in a clinical setting. Mass spectrometry has also been used to measure ADMA concentrations. However, this method is also inappropriate for a simple and reliable clinical assay because it is complicated and requires the use of expensive instrumentation.

Researchers have tried several approaches to assay asymmetric methylarginines using DDAH as a binding partner. These included studying how ADMA binds to inactive mutations of DDAH and how the binding of ADMA to DDAH could be improved by generating mutations around the active site. However, none of these were approaches were successful (unpublished observations). Firstly, the researchers tried to get a Kd by equilibrium dialysis and calorimetry for the inactive mutations (C249S, H162A and E114Q) without success. In the calorimetry experiments saturation was never reached even with very high concentrations of ADMA (10 mM). Secondly, the researchers tried to improve the binding of ADMA to DDAH through mutagenesis. Three more mutations were generated in E114 and around this residue. These were E114D, E114D+A115G, L18H (residue in the loop) and P80A. These four mutations increased the Km of ADMA (decreased the affinity for ADMA) compared to the wild type DDAH. Other mutations around the active site were also generated: L161F, G116A, G116V and G116T. In general all these mutations either increased the Km for ADMA (decreased affinity) or they resulted in the DDAH not being expressed properly.

Thirdly, the researchers tried to calculate a Kd for ADMA of DDAH using nuclear magnetic resonance imaging using the C249S or H162A mutant. However, this was unsuccessful because, during the binding to ADMA, the inactive protein could not be saturated with ADMA or there were problems associated with the residues in the binding site. Fourthly, the researchers tried a fluorophore (fluoresceine 5 maleimide or acrylodan) approach to measure the Kd for ADMA. This approach used the inactive mutant C249S with other mutations in the loop (S20C and S21C) and involved incorporating the two fluorophores into the protein (bound to S20C or S21C). There was no change in the fluorescence spectrum of the protein when it was incubated with ADMA. This suggested that the loop moves too fast or these residues are not relevant for the binding of ADMA. Finally, the researchers generated four mutations with tryptophan: V15W and D16W (in the loop) as well as F58W and E90W (close to the active site). These four residues move in the NMR spectrum when inhibitors bind to the protein. However, no changes were observed using fluorimetry when the proteins were incubated with known inhibitors (including ADMA). Hence, DDAH is unsuitable for use in a competitive binding assay for measuring ADMA.

### SUMMARY OF THE INVENTION

The present inventors have identified that asymmetric methylarginines reversibly bind to NOS polypeptides and that NOS polypeptides can be used in a competitive binding assay to determine the presence and/or amount of an asymmetric methylarginine.

Accordingly, the present invention provides a method of determining the presence and/or amount of an asymmetric methylarginine in a sample, the method comprising:

(a) contacting the sample with a nitric oxide synthase (NOS) polypeptide in the presence of a detectably labelled species under conditions which permit the asymmetric methylarginine and detectably labelled species to bind to the NOS polypeptide; and

(b) determining the amount or presence of the detectably labelled species bound to the NOS polypeptide. Typically, the amount of the detectably labelled species bound to the NOS polypeptide is compared with the amount of the detectably labelled species bound to or expected to be bound to the NOS polypeptide in the absence of the sample to determine the presence and/or amount of the asymmetric methylarginine in the sample.

Suitable detectably labelled species are compounds which contain a section of their structure which is chemically similar to arginine or asymmetric methylarginine.

The invention also provides:
use of a NOS polypeptide to determine the presence and/or amount of an asymmetric methylarginine in a sample; and
a kit for determining the presence and/or amount of an asymmetric methylarginine in a sample, comprising:
   a NOS polypeptide; and
   a detectably labelled species.

### Description of the Sequences Mentioned Herein

SEQ ID NO: 1 shows the amino acid sequence of the bacillus subtilis nitric oxide synthase oxygenase domain (bsNOS) as described in Yamamoto et al., Gene 194 (2), 191-199 (1997).

SEQ ID NO: 2 shows the amino acid sequence of the bacillus subtilis nitric oxide synthase oxygenase domain (bsNOS) as described in Pant et al., Biochemistry 41 (37), 11071-11079 (2002).

### BRIEF DESCRIPTION OF THE DRAWING

FIG. 1 shows L-NMMA binding to J774 cells. The Y-axis shows [¹⁴C]L-NMMA bound to iNOS lysate (CPM). A=J774 stimulated, B=J774 stimulated+L-NMMA, C=control and D=control+L-NMMA.

FIG. 2 shows L-NMMA binding to bsNOS. The Y-axis shows [¹⁴C]L-NMMA bound to bsNOS(CPM). A=Control and B=excess L-NMMA.

FIG. 3 shows L-NMMA binding to bsNOS. The Y-axis shows [¹⁴C]L-NMMA bound to bsNOS(CPM). A=Control and B=excess L-NMMA.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a method” includes “methods”, reference to “a polypeptide” includes a mixture of two or more such polypeptides, reference to “a label” includes two or more such labels, and the like.

All publications, patents and patent applications cited herein, whether supra or infra, are hereby incorporated by reference in their entirety.

### Competitive Binding Assay for Asymmetric Methylarginines

The method of the invention uses a competitive binding approach to detect the presence of an asymmetric methylarginine in a sample and/or determine the amount of the asymmetric methylarginine. The general technique of such competitive binding assays is well known in the art. They give signals which decrease as the concentration of the target analyte increases. Furthermore, methods for adapting these approaches to give signals which increase with increasing concentration of analyte are known. The methods can also be further adapted into a ‘sandwich’ format using a second binding molecule, such as an antibody, to recognise the structural changes which take place when the asymmetric methylarginine binds to the NOS polypeptide. The method involves determining the effect of the asymmetric methylarginine in the sample on the binding of a detectably labelled species to a NOS polypeptide. The invention therefore relates to the use of a NOS polypeptide to detect the presence of an asymmetric methylarginine in a sample and/or determine the amount of the asymmetric methylarginine.

An asymmetric methylarginine is an asymmetric methylated arginine. An asymmetric methylarginine is an arginine in which the terminal nitrogen atoms on the side group have been differentially methylated. The asymmetric methylated arginine may contain one or more methyl groups, such as one, two or three methyl groups. The asymmetric methylarginine preferably contains one or two methyl groups. The asymmetric methylarginine preferably has the following formula (I):

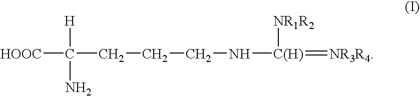

wherein:
R₁, R₂, R₃ and R₄ are each independently H, methyl or C₂₋₆ alkyl such as ethyl, butyl, pentyl or hexyl;
at least one of R₁, R₂, R₃ and R₄ is methyl;
[symbol] is a double or single bond where (H) is present if it is a single bond; and
NR₁R₂ is not the same as NR₃R₄.

The asymmetric methylarginine is preferably asymmetric dimethylarginine (ADMA) or N^{G}-monomethyl-L-arginine (L-NMMA).

When the sample is contacted with the detectably labelled species and NOS polypeptide, the asymmetric methylarginine in the sample competes with the detectably labelled species for binding to the NOS polypeptide. The components of the competitive binding assay may be contacted with each other in any order. The detectably labelled species is preferably contacted with the NOS polypeptide before the sample is contacted with the NOS polypeptide. The sample may be contacted with the detectably labelled species before contacting it with the NOS polypeptide.

After contact, bound, detectably labelled species is detected or measured using a method appropriate for the given label (for example scintillation counting, enzyme assay, fluorescence or electrochemistry). Methods using fluorescence or electrochemistry are particularly suitable for devices that are intended for near-patient use, for example in the home or doctor's office or at the bedside. Fluoresence or electrochemistry measurements can easily be incorporated into assay strip devices that are used once only and read in a suitably configured reader, before the strip is disposed of. Suitable labels for use in accordance with the invention are discussed in more detail below. A change in binding of the detectably labelled species to the NOS polypeptide is indicative of the presence of the asymmetric methylarginine in the sample. The extent of the change in binding is indicative of the amount of the asymmetric methylarginine in the sample.

Any suitable binding assay format can be used to monitor binding and detect any effect. The effect is measured as a decrease in the binding between a detectably labelled species and a NOS polypeptide. For example, a decrease of at least 5%, at least 10%, at least 20%, at least 30%, at least 40%, at least 50%, at least 60%, at least 70% or at least 80% in the binding between a detectably labelled species and a NOS polypeptide measured in any given assay indicates that an asymmetric methylarginine is present in the sample. To calibrate the assay, control competition reactions using increasing known concentrations of an unlabelled species in place of the sample may be carried out. The resultant calibration curve can be stored in the instrument used to read the assay, such that the assay result is immediately obtained.

The amount of the detectably labelled species bound to the NOS polypeptide may be measured directly or indirectly. A direct measurement may be carried out by removing assay mixture containing the unbound detectably labelled asymmetric methylarginine and measuring the amount of label that is in the NOS polypeptide fraction. Alternatively, the amount of labelled reference compound bound to the product could be determined indirectly by measuring the amount of label remaining in the assay solution after removal of the NOS polypeptide fraction, which will be inversely related to the amount that has bound to the product. Further alternatively, the assay could be incorporated into a microfluidic system which is configured so that the only requirement is to apply a drop of blood or plasma to an assay strip.

In the competitive binding assay system, the NOS polypeptide may be immobilised on a solid support, such as particles, a porous matrix like nitrocellulose, or the surface of a sample well or microfluidic device, or may be in solution. The use of immobilised NOS polypeptide has the advantage that, after the binding reaction is complete, the NOS polypeptide/detectably labelled species complex may be separated from the detectably labelled species that remains in solution by simply removing the solution away from the solid support. Examples of such assays are lateral flow or microfluidic type devices. If, on the other hand, the product is not immobilised during the assay but rather is in solution, then it will generally be necessary to devise a means for separating the NOS polypeptide/detectably labelled species complex from the uncomplexed detectably labelled species before measuring the amount of label. Such separation could be achieved, for example, by precipitating the product using an antibody to the NOS polypeptide or by using a non-specific precipitation technique, or by capture of the NOS polypeptide onto a surface that has been previously activated by binding of an antibody to the NOS polypeptide.

Suitable solid supports are well known in the art and include plates, such as mulit well plates, filters, membranes, beads, chips, pins, dipsticks and porous carriers. The NOS polypeptide may be immobilised on a support using an antibody or via other technologies which are known in the art.

The methods of the invention are carried out under conditions which allow the asymmetric methylarginine and detectably labelled species to bind to the NOS polypeptide. These conditions are, for example, the temperature, salt concentration, pH and protein concentration under which a asymmetric methylarginine binds to a NOS polypeptide. Exact binding conditions will vary depending upon the nature of the assay. However, preferred conditions will generally include physiological salt concentration (approximately 85-95 mM) and pH (about 7.0 to 8.0). Temperatures for binding may vary from 4° C. to 37° C., but is preferably between 15-25° C. The concentration of reactants in the binding assay will also vary, but will preferably be from about 0.1 μM to about 10 μM.

### Nitric Oxide Synthase (NOS) Polypeptide

The phrase “nitric oxide synthase (NOS) polypeptide” is intended to include all naturally occurring forms of eNOS, iNOS and nNOS as well as variants which retain the ability to bind an asymmetric methylarginine, for example variants produced by mutagenesis techniques. The NOS polypeptide may be of mammalian origin, for example rodent (including rat and mouse) or primate (such as human). The NOS polypeptide may be encoded by any of the sequences shown in the Table below or a variant of any one of those enzymes that retains the ability to bind an asymmetric methylarginine.

| Sequence | GenBank Accession No. | Coding sequence |
|---|---|---|
| Human iNOS | X73029 | 226-3687 |
| Human nNOS | U17327 | 686-4990 |
| Human eNOS | M95296 | 21-3632 |
| Rat iNOS | NM_012611 | 186-3629 |
| Rat nNOS | NM_052799 | 102-4493 |
| Rat eNOS | NM_021838 | 7-3615 |
| Mouse iNOS | NM_008712 | 99-4388 |
| Mouse nNOS | NM_010927 | 185-3619 |
| Mouse eNOS | NM_008713 | 22-3630 |

The NOS polypeptide may also be bacterial. Preferably, the NOS polypeptide comprises the bacillus subtilis nitric oxide synthase oxygenase domain (bsNOS; SEQ ID NO: 1 or 2) or a variant thereof that retains the ability to bind an asymmetric methylarginine.

A variant of a NOS is any polypeptide variant of a NOS which retains its ability to bind an asymmetric methylarginine, preferably a fragment of a NOS. A fragment may be of any length, so long as it retains the ability to bind an asymmetric methylarginine. A fragment preferably comprises the oxygenase domain of a NOS.

Typically, the binding affinity for an asymmetric methylarginine of such a variant is substantially the same as that of the wild-type NOS. Alternatively, the binding affinity for an asymmetric methylarginine may be greater or less than that of the wild-type NOS. For example, a variant may have a binding affinity for an asymmetric methylarginine which is at least 95%, at least 90%, at least 85%, at least 80%, at least 75%, or at least 70% of that of the wild type NOS. Alternatively, the binding affinity for an asymmetric methylarginine of the variant may be at least 105%, at least 110%, at least 120%, or at least 130% of that of the wild type NOS. For instance, the binding affinity for an asymmetric methylarginine of a variant of a NOS may be from 95% to 105%, from 90% to 110%, from 85% to 120%, from 80% to 130%, from 75% to 140% or from 70% to 150% of that of the wild-type. The affinity constant for the interaction between a variant of a NOS and an asymmetric methylarginine is typically from 1×10⁻⁶ M to 1×10⁻²M. For example, the affinity constant may be from 1×10⁻⁷M to 1×10⁻¹¹M or from 1×10⁻⁸M to 1×10¹¹M.

A variant of a NOS useful in the invention comprises a sequence substantially similar to that of a naturally occurring form or wild-type form of a NOS. Thus, a variant of a NOS will generally have at least 60%, at least 70%, at least 80%, at least 90%, at least 95%, at least 98% or at least 99% sequence identity to the relevant NOS sequence, calculated over the full length of those sequences. The identity may be calculated on the basis of nucleotide or amino acid identity (sometimes referred to as “hard homology”). For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

A BLAST analysis is preferably used for calculating identity. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

A variant of a NOS may have, for example, amino acid substitutions, deletions or additions compared to the wild-type. At least 1, at least 2, at least 3, at least 5, at least 10 or at least 20 amino acid substitutions or deletions, for example, may be made, up to a maximum of 100 or 50 or 30. For example, from 1 to 100, from 2 to 50, from 3 to 30, or from 5 to 15 amino acid substitutions or deletions may be made. Typically, if substitutions are made, the substitutions will be conservative substitutions, for example according to the following Table. Amino acids in the same block in the second column and preferably in the same line in the third column may be substituted for each other. Deletions are preferably deletions of amino acids from one or both ends of the sequence of the NOS protein. Alternatively, deletions are of regions not involved in the interaction with an asymmetric methylarginine.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Fragments of a NOS which retain the ability to bind an asymmetric methylarginine are preferably used. Such fragments may be from 250 to 1300 amino acids in length and are preferably at least 300, 330, 350, 400, 450, 500, 550, 650, 700, 720, 800, 900 or 1000 amino acids long.

Any of the NOS polypetides useful in the invention may further be chemically-modified to form a derivative. Derivatives include polypeptides that have lipid extensions or have been glycosylated. Suitable derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemically modified polypeptides are those which contain one or more naturally occurring amino acid derivatives of the twenty standard amino acids. For example, 4-hydroxyproline may be substituted for proline or homoserine may be substituted for serine.

Derivatives also include polypeptides that have been detectably labelled. Detectably labelled polypeptides have been labelled with a labelling moiety that can be readily detected. Examples of labelling moieties include, but are not limited to, radioisotopes or radionucleodtides, fluorescent molecules such as green fluorescent protein (GFP), electrochemically redox active molecules such as ferrocene derivatives, phosphorescent molecules, electron-dense reagents, quenchers of fluorescence, enzymes, affinity tags, epitope tags, antibodies, polynucleotides and polypeptides such as biotin. Suitable radioisotopes include ¹⁴C, ³H, ¹²⁵I, ³⁵S and ³²P. Affinity tags are labels that confer the ability to specifically bind a reagent onto the labelled molecule. Examples include, but are not limited to, biotin, histidine tags and glutathione-S-transferase (GST). Labels may be detected by, for example, spectroscopic, photochemical, radiochemical, biochemical, immunochemical chemical or electrochemical methods that are known in the art.

Any of the NOS polypeptides useful in the invention may also comprise additional amino acids or polypeptide sequences. Any of the NOS polypeptides useful in the invention may comprise additional polypeptide sequences such that they form fusion proteins. The additional polypeptide sequences may be fused at the amino terminus, carboxy terminus or both the amino terminus and the carboxy terminus of the NOS peptide. Examples of fusion partners include, but are not limited to, GST, maltose binding protein, alkaline phosphatates, thiorexidin, GFP, histidine tags and epitope tags (for example, Myc or FLAG). The additional sequence may perform any known function. Typically, it may be added for the purpose of providing a carrier polypeptide, by which the NOS polypeptide can be, for example, affixed to a label, solid support or carrier. Thus the first component for use in the invention may be in the form of a fusion polypeptide which_comprises heterologous sequences. Indeed, in practice it may often be convenient to use fusion polypeptides. This is because fusion polypeptides may be easily and cheaply produced in recombinant cell lines, for example recombinant bacterial or insect cell lines. Fusion polypeptides may be expressed at higher levels than the wild-type NOS protein or variant thereof. Typically this is due to increased translation of the encoding RNA or decreased degradation. In addition, fusion polypeptides may be easy to identify and isolate. Typically, fusion polypeptides will comprise a polypeptide sequence as described above and a carrier or linker sequence. The carrier or linker sequence will typically be derived from a non-human, preferably a non-mammalian source, for example a bacterial source. This is to minimize the occurrence of non-specific interactions between heterologous sequences in the fusion polypeptide and the asymmetric methylarginine, which is the target of the NOS polypeptide.

The NOS polypeptide may be expressed using recombinant DNA techniques. For example, suitable polypeptides may be expressed in, for example, bacterial or insect cell lines (see, for example, Munger et al., 1998, Molecular Biology of the Cell, 9, 2627-2638). Typically, a recombinant NOS polypeptide can be produced by expression in E. coli. Recombinant polypeptides are produced by providing a polynucleotide encoding a NOS polypeptide. Such polynucleotides are provided with suitable control elements, such as promoter sequences, and provided in expression vectors and the like for expression of the NOS polypeptide. Suitable polynucleotides may be isolated biochemically from any suitable bacteria.

Alternatively, the NOS polypeptide used in the assays may be obtained from mammalian or bacterial cellular extracts. The NOS polypeptide can be obtained from cells that express NOS proteins endogenously or through the use of recombinant techniques. For example, the NOS polypeptide may be obtained from endothelial cells.

Alternatively, the NOS polypeptide may be chemically synthesized. Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). In general, solid-phase synthesis methods comprise the sequential addition of one or more amino acid residues or suitably protected amino acid residues to a growing polypeptide chain.

### Sample

The sample for analysis may be any suitable biological sample. The invention is typically carried out in vitro on a sample obtained from a patient. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs.

The sample may be from a subject at risk of, or suffering from, endothelial dysfunction and cardiovascular disease. Preferably, the sample is from a subject at risk of, or suffering from, renal failure, hypercholesterolaemia, hypertension, diabetes, hyperhomocystinaemia and overt atherosclerosis, type 2 diabetes, heart failure or pulmonary hypertension.

Preferably, the sample is from a subject at risk of, or suffering from, pre-eclampsia or whose fetus is at risk of, or suffering from, intrauterine growth restriction (IUGR). The sample may therefore be from a pregnant woman or her fetus. Typically the woman or fetus is at a stage of pregnancy from 4 to 25 weeks gestation. The woman or fetus may be at a stage of pregnancy from 23 to 25 weeks gestation. Preferably the woman or fetus is at a stage of pregnancy from 10 to 25 weeks gestation and more preferably from 15 to 25 weeks gestation. Typically the woman does not have pre-eclampsia or displays no symptoms of pre-eclampsia but is suspected as being at risk or selected as being predisposed to developing pre-eclampsia. Typically the fetus does not have IUGR or displays no symptoms of IUGR but is suspected as being at risk or selected as being predisposed to developing IUGR. Risk factors that increase susceptibility to developing pre-eclampsia or IUGR typically include Afro-Caribbean ancestry, nullparity or first pregnancy with a partner, multiple gestations, hypertension, diabetes, genetic predisposition to or family history of pre-eclampsia or eclampsia, obesity, hypercholesterolaemia and smoking. Typically the pregnant woman is a smoker.

The sample is typically processed prior to being assayed, for example by centrifugation or by passage through a membrane that filters out red blood cells, yielding plasma. The sample may be measured immediately upon being taken. The sample may also be typically stored prior to assay, preferably below −70° C.

### Detectably Labelled Species

The species that is detectably labelled is any compound that is able to bind to the active binding region of a NOS polypeptide. The species is preferably able to bind to the region of NOS that binds arginine. The species is preferably a competitive inhibitor of NOS. Competitive inhibitors of NOS are compounds which contain a section of their structure which is chemically similar to arginine or asymmetric methylarginine. For example, the species that is detectably labelled may be any of the asymmetric methylarginines discussed above. The species that is detectably labelled may be the same as or different to the asymmetric methylarginine in the sample to be assayed. The species that is detectably labelled is preferably the same asymmetric methylarginine that is in the sample to be assayed. The species that is detectably labelled is preferably asymmetric dimethylarginine (ADMA), N^{G}-monomethyl-L-arginine (L-NMMA) or arginine.

Examples of labelling moieties include, but are not limited to, radioisotopes or radionucleodtides, fluorescent molecules or moieties such as green fluorescent protein (GFP), phosphorescent molecules, electron-dense reagents, quenchers of fluorescence, enzymes, affinity tags, epitope tags, antibodies, polynucleotides, polypeptides such as biotin, magnetic species, particulate labels such as colloidal metal sols, polymeric particles containing visible or fluorescent dye, dye sols and electrochemically redox active molecules. Suitable radioisotopes include ¹⁴C, ³H, ¹²⁵I, ³⁵S and ³²P. Suitable electrochemically active redox molecules include ferrocene derivatives. Affinity tags are labels that confer the ability to specifically bind a reagant onto the labelled molecule. Examples include, but are not limited to, biotin, histidine tags and glutathione-S-transferase (GST). The species is preferably detectably labelled with an electrochemically active redox molecule or a fluorescent or phosphorescent molecule. Labels may be detected by, for example, spectroscopic, photochemical, radiochemical, biochemical, immunochemical chemical or electrochemical methods that are known in the art.

### Kits

The invention also provides various kits for determining the presence and/or amount of an asymmetric methylarginine in a sample. These kits comprise:
a NOS polypeptide; and
a detectably labelled species.

The kit may additionally comprise one or more other reagents or instruments which enable any of the embodiments of the method mentioned above to be carried out. Such reagents or instruments include one or more of the following: suitable buffer(s) (aqueous solutions), means to obtain a sample from the subject (such as a vessel or an instrument comprising a needle) or a support comprising wells on which quantitative reactions can be done, or a microfluidic device incorporating binding surfaces on which quantitative reactions can be carried out. Reagants may be present in the kit in a dry state such that a fluid sample resuspends the reagents. The kit may, optionally, comprise instructions to enable the kit to be used in the method of the invention or details regarding which patients the method may be used for. The kit may, optionally, comprise an antagonist of ADMA activity. The antagonist is preferably L-arginine. The kit may, optionally, contain reagents, such as arginine deiminase, for removing arginine from the sample, thereby avoiding possible complications in the assay system caused by competition between asymmetric methylarginine and unlabelled arginine that is naturally present in the sample.

The present invention is described with reference to the following, non-limiting Examples:

### EXAMPLES

Initial studies indicated that millimolar concentrations of ADMA were necessary in order to get binding to DDAH. Various studies have shown that both ADMA and L-NMMA compete with arginine to inhibit nitric oxide synthase and IC₅₀ values have been calculated for the 3 enzymes to be in the range 0.18-6.2 μM (Olken N M et al., Biochem Biophys Res Commun. 1991; 177:828-33; Frey C et al., J Biol. Chem. 1994; 269:26083-91; Pollock J S et al., Proc Natl Acad Sci USA. 1991; 88:10480-4; and Griffith O W et al., Methods Enzymol. 1996; 268:375-92) where the Km for arginine ranges from 7-19 μM. Furthermore ADMA and L-NMMA inhibition of NOS appear to be equipotent. Therefore a decision was made to detect L-NMMA and ADMA using NOS.

### Example 1

Macrophages (J774 cells) were stimulated with a cytokine cocktail (LPS/IFN/TNF) for 24 h, to induce iNOS expression, and cell lysates were collected. The induction of iNOS was confirmed by measurement of NOx generation in culture media. Low molecular weight molecules were removed from lysates using 30000 MW cut-off filters. Lysates were incubated on 3000 MW filters with [¹⁴C]L-NMMA for 15 min (∀ 1:M L-NMMA), washed 3× with cold TRIS buffer and filters counted.

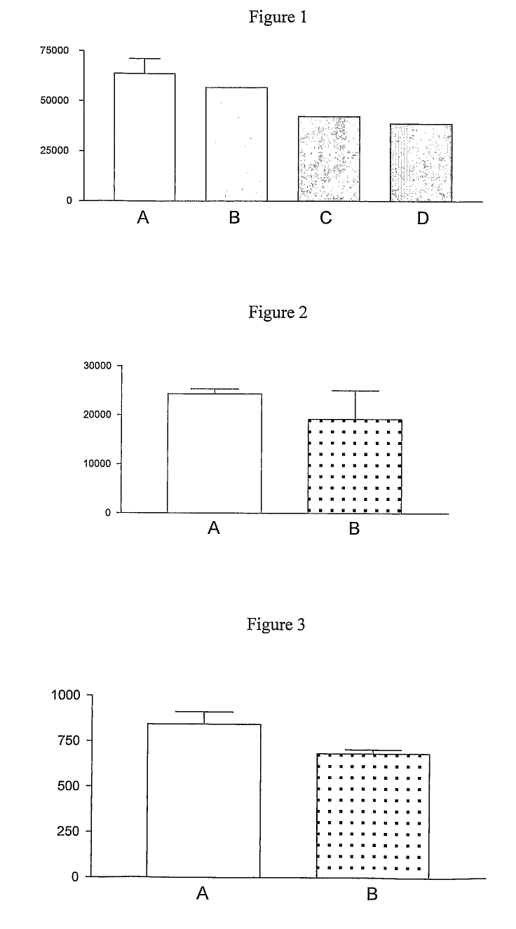

These preliminary studies indicated that the [14C]-NMMA bound to NOS and that this could be competed out by excess 1000-fold L-NMMA (FIG. 1). There are issues for binding [¹⁴C]L-NMMA to cell lysates which also contain DDAH therefore it was decided to use a recombinant bacterial NOS.

### Example 2

A bacillus subtilis nitric oxide synthase oxygenase domain (“fragment”) was PCR amplified from genomic DNA with Nde1 and BamH1 sites added at the 5′ and 3′ ends respectively and cloned into pET15b vector. The bsNOS fragment corresponded to SEQ ID NO: 2 and was purified as previously described (Pant et al., Biochemistry; 2002; 41:11071-11079).

### Example 3

[¹⁴C]L-NMMA (3.75 nmol) was incubated with the bsNOS fragment (10 μmol) on 30000 MW cut off filters in the presence and absence (Control) of 1000-fold excess unlabelled L-NMMA. These preliminary studies indicated that the [¹⁴C]-NMMA bound to NOS and that this could be competed out by excess 1000-fold L-NMMA (FIG. 2).

### Example 4

### bsNOS Binding to [¹⁴C]L-NMMA

A modified method previously described for binding [3H]nitroarginine to mammalian NOS was used to ensure rapid washes of the bsNOS fragment complexes and ligand to reduce time for dissociation (Liu Q and Gross S S, Methods Enzymol. 1996; 268:311-24). [¹⁴C]L-NMMA (50 μmol −20000 counts total) was incubated with the bsNOS fragment (10 μmol) in assay buffer containing: 50 mM Tris pH 7.6; 50 uM BH4, 0.5 mM DTT. Reactants were incubated for 15 min at room temperature and NOS-bound [¹⁴C]L-NMMA was separated from free [¹⁴C]L-NMMA using PVDF filters and 3×1 ml rapid washes in cold Tris pH 7.6. Background was determined by the addition of 1 mM L-NMMA to bsNOS in assay buffer. Filters were added to 5 ml scintillation cocktail and counted in scintillation counter (Beckmann).

The bsNOS fragment appeared to bind to [¹⁴C]L-NMMA and with the binding reduced to background in the presence of L-NMMA (1 mM) (FIG. 3).

1. A method of determining the presence and/or amount of an asymmetric methylarginine in a sample, the method comprising:
(a) contacting the sample with a nitric oxide synthase (NOS) polypeptide in the presence of a detectably labelled species under conditions which permit the asymmetric methylarginine and detectably labelled species to bind to the NOS polypeptide; and
(b) determining the amount or presence of the detectably labelled species bound to the NOS polypeptide.

2. A method according to claim 1, wherein the asymmetric methylarginine in the sample is asymmetric dimethylarginine (ADMA) or N^{G}-monomethyl-L-arginine (L-NMMA).

3. A method according to claim 1, wherein the NOS polypeptide is a fragment of a wild-type NOS that retains its ability to bind the asymmetric methylarginine.

4. A method according to claim 1, wherein the NOS polypeptide comprises SEQ ID NO: 1 or 2 or a variant thereof that retains the ability to bind an asymmetric methylarginine.

5. A method according to claim 1, wherein the sample is blood, plasma, serum, urine or amniotic fluid.

6. A method according to claim 1, wherein the sample is from a subject at risk of, or suffering from, renal failure, hypercholesterolaemia, hypertension, diabetes, hyperhomocystinaemia and overt atherosclerosis, type 2 diabetes, heart failure or pulmonary hypertension.

7. A method according to claim 1, wherein the sample is from a subject at risk of, or suffering from, pre-eclampsia or whose fetus is at risk of, or suffering from, intrauterine growth restriction (IUGR).

8. A method according to claim 1, wherein the species that is detectably labelled is an arginine derivative.

9. A method according to claim 1, wherein the species that is detectably labelled is the same as the asymmetric methylarginine in the sample.

10. A method according to claim 1, wherein the species is detectably labelled with a) a fluorescent or phosphorescent molecule or b) an electrochemically active redox molecule.

11. (canceled)

12. A method according to claim 10, wherein electrochemically active redox molecule is a ferrocene derivative.

13. (canceled)

14. A kit for determining the presence and/or amount of an asymmetric methylarginine in a sample, comprising:
a NOS polypeptide; and
a detectably labelled species.

15. A kit according to claim 14, wherein the NOS polypeptide is immobilised on a solid support.

16. A kit according to claim 14, wherein the asymmetric methylarginine in the sample is asymmetric dimethylarginine (ADMA) or N^{G}-monomethyl-L-arginine (L-NMMA).

17. A kit according to claim 14, wherein the NOS polypeptide is a fragment of a wild-type NOS that retains its ability to bind the asymmetric methylarginine.

18. A kit according to claim 14, wherein the NOS polypeptide comprises SEQ ID NO: 1 or 2 or a variant thereof that retains the ability to bind an asymmetric methylarginine.

19. A kit according to claim 14, wherein the sample is blood, plasma, serum, urine or amniotic fluid.

20. A kit according to claim 14, wherein the sample is from a subject at risk of, or suffering from, a) renal failure, hypercholesterolaemia, hypertension, diabetes, hyperhomocystinaemia and overt atherosclerosis, type 2 diabetes, heart failure or pulmonary hypertension or b) pre-eclampsia or whose fetus is at risk of, or suffering from, intrauterine growth restriction (IUGR).

21. (canceled)

22. A kit according to claim 14, wherein the species that is detectably-labelled is an arginine derivative and/or b) the same as the asymmetric methylarginine in the sample.

23. (canceled)

24. A kit according to claim 14, wherein the asymmetric methylarginine is detectably labelled with a fluorescent or phosphorescent molecule.

25. A kit according to claim 14, wherein the asymmetric methylarginine is detectably labelled with an electrochemically redox active molecule.

26. A kit according to claim 25, wherein the electrochemically redox active molecule is a ferrocene derivative.

27. A kit according to claim 14, wherein the kit includes a means for decreasing the concentration of arginine naturally present in the sample.

28. A kit according to claim 27, wherein the means comprises contacting the sample with arginine deiminase.

29. A kit according to claim 14, wherein the kit comprises a microfluidic device.


SEQUENCE LISTING (2 sequences)

SEQ ID NO: 1 - protein, 336 aa, Bacillus subtilis
MKDRLADIKSEIDLTGSYVHTKEELEHGAKMAWRNSNRCIGRLFWNSLNVIDRRDVRTKEEVRDALFHHIETATNNGKIRPTITIFPPEEKGEKQVEIWNHQLIRYAGYESDGERIGDPASCSLTAACEELGWRGERTDFDLLPLIFRMKGDEQPVWYELPRSLVIEVPITHPDIEAFSDLELKWYGVPIISDMKLEVGGIHYNAAPFNGWYMGTEIGARNLADEKRYDKLKKVASVIGIAADYNTDLWKDQALVELNKAVLHSYKKQGVSIVDHHTAASQFKRFEEQEEEAGRKLTGDWTWLIPPISPAATHIFHRSYDNSIVKPNYFYQDKPYE

SEQ ID NO: 2 - protein, 363 aa, Bacillus subtilis
GSHMEILWNEAKAFIAECYQELGKEEEVKDRLDSIKSEIDRTGSYVHTKEELEHGAKMAWRNSNRCIGRLFWNSLNVIDRRDVRTKEDVRDALFHHIETATNNGKIRPSITIFPPEEKGEKQVEIWNHQLIRYAGYESDGERIGDPASRSLTAACEQLGWRGERTDFDLLPLIFRMRGDEQPVWYELPRSLVIEVPITHPDIEAFSDLELKWYGVPIISDMKLEVGGIHYNAAPFNGWYMGTEIGARNLADEKRYDKLKKVASVIGISTNYNTDLWKDQALVELNKAVLYSYKKQGVSIVDHHTAASQFKRFEEQEEEAGRKLTGDWTWLIPPISPAATHIFHRSYDNSIVKPNYFYQDKPYE